(19) United States
(12) Patent Application Publication
(10) Pub. No.: US 2012/0016217 A1
(43) Pub. Date: Jan. 19, 2012

(54) BIOSENSOR FOR HEALTH MONITORING AND USES THEREOF

(75) Inventors: Rohit SRIVASTAVA, Mumbai (IN); Abhijeet Balwantrao Joshi, Thane (IN)

(73) Assignee: Indian Institute of Technology Bombay

(21) Appl. No.: 12/837,218

(22) Filed: Jul. 15, 2010

(51) Int. Cl. A61B 5/1473; A61B 5/1459; G01N 27/26

(52) U.S. Cl. 600/317; 205/792; 600/309; 600/341

(57) ABSTRACT

Disclosed are methods, compositions and kits pertaining to detecting and/or measuring biological analytes. In certain embodiments the disclosure relates to detection and measurement of lactate, pyruvate, oxygen and/or hydrogen peroxide. In some aspects and embodiments provided are biosensors that may be or include nanoparticles, microparticles and/or nano-in-micro matrices. The methods and compositions disclosed may have uses in purpose of diagnosis and treatment of diseases, fitness and health monitoring in sports, in space medicine, in food applications and the food industry, in pharmaceutical applications and the pharmaceutical industry, for fermentation monitoring, and in polymer manufacturing applications. In some embodiments and aspects, the disclosure relates to minimally invasive biosensors and/or biosensors that may be used for continuous monitoring.

### TECHNICAL FIELD

This disclosure relates generally to methods, kits and compositions pertaining to detecting and/or measuring biological analytes. In certain embodiments the disclosure relates to detection and measurement of lactate, pyruvate, oxygen and/or hydrogen peroxide.

### BACKGROUND

The following description is provided to assist the understanding of the reader. None of the information provided or references cited is admitted to be prior art.

Development of biosensors has opened up new avenues for continuous monitoring of biological analytes. Current commercially available lactate biosensors include the YSI 1500® sport lactate analyzer, and the Roche Accutrend lactate meter LCM®. Compositions including microparticles and nanoparticles have been used as biosensors for the detection of analytes, for example, Lee et al. (In vivo imaging of hydrogen peroxide with chemiluminescent nanoparticles, Nature Letters p. 1-5, (2007)) discloses the use of nanoparticles to detect hydrogen peroxide in vivo. The incorporation of enzymes into microparticles and nanoparticles can be useful for generating biosensors and for the emission of detectable signals. In this regard, Abhijeet B. Joshi and Rohit Srivastava (Polyelectrolyte Coated Calcium Carbonate Micro-particles as Templates for Enzyme Encapsulation, Advanced Science Letters Vol. 2(3), p. 1-8 (2009)) discloses calcium carbonate microcapsules for enzyme encapsulation.

### SUMMARY

The compositions and methods described herein are based at least in part on the discovery that minimally invasive biosensors can effectively detect and measure biological analytes such as, for example, lactate, pyruvate, oxygen and/or hydrogen peroxide.

In one aspect, the present technology provides biosensors, nanoparticles, microparticles and/or nano-in-micro matrices. In certain illustrative embodiments, the compositions and methods allow for minimally invasive measurements of biological analytes such as, for example, lactate, pyruvate, oxygen and/or hydrogen peroxide and may also provide the ability of continuous monitoring.

In certain embodiments, a biosensor (for example a nanoparticle, a microparticle and/or a nano-in-micro matrix) of the present technology includes at least one fluorophore, for example the fluorophore may be pentacene or rubrene. In some embodiments, a biosensor of the present technology emits fluorescence, luminescence, light, and/or chemiluminescence in the presence of an analyte (for example, lactate, pyruvate, oxygen or hydrogen peroxide). In certain embodiments the biosensor emits fluorescence, luminescence, light, and/or chemiluminescence in the presence of the analyte when excited with light, for example light at a different wavelength than the emitted fluorescence, luminescence, light, and/or chemiluminescence. In certain embodiments the biosensor emits fluorescence, luminescence, light, and/or chemiluminescence in the presence of the analyte when excited with light at 630 nm. In some embodiments, the biosensor emits light in the near infra red (NIR) range (600 nm to 1100 nm). In some embodiments the biosensor emits light at about 630 nm; or at about 650 nm; or at about 648 nm; or at about 653 nm. In some embodiments the biosensor emits light at a wavelength above 600 nm; or above 625 nm; or above 645 nm; or at above 650 nm.

In certain embodiments the biosensor includes one or more of polystyrene sulphonate (PSS), poly allyl amine hydrochloride (PAH), polyacrylic acid (PAA), chondroitin sulphate, collagen, dextran sulphate, polyethylene imine (PEI), chitosan, alginate.

In some embodiments, a biosensor (for example a nanoparticle, a microparticle and/or a nano-in-micro matrix) of the present technology includes a lactic acid catalyzing enzyme, for example, lactate oxidase (Lox), lactate dehydrogenase, and/or lactate monooxygenase. In some embodiments a biosensor (for example a nanoparticle, a microparticle and/or a nano-in-micro matrix) of the present technology includes a peroxalate ester or polymer. In some related embodiments the peroxalate ester or polymer may emit light at 630 nm in the presence of hydrogen peroxide. In some embodiments, a biosensor (for example a nanoparticle, a microparticle and/or a nano-in-micro matrix) of the present technology includes at least one oxygen sensitive dye. In some related embodiments the oxygen sensitive dye is a near infra red dye; in some embodiments the oxygen sensitive dye is ruthenium or a ruthenium based dye, a porphyrin, pentacene platinum octa-ethyl porphyrin (PtOEP), rudppy or PtOEP ketone or any fluorophore like pentacene, Aniline suphonic acid (ANS), rubrene, perylene. In certain embodiments, a biosensor, nanoparticle, microparticle and/or nano-in-micro matrix of the present technology includes an oxygen insensitive dye; in some embodiments a biosensor (for example a nanoparticle, a microparticle and/or a nano-in-micro matrix) of the present technology includes both an oxygen sensitive dye and an oxygen insensitive dye. In some embodiments the oxygen insensitive dye is pentacene, rubrene, ANS, Alexa fluor, tetra methyl rhodamine iso-thiocyanate (TRITC), or rhodamine. In certain embodiments the oxygen insensitive dye is used as a standard or reference dye; for example it may be used to standardize values from other dyes.

In some embodiments, a biosensor (for example a nanoparticle, a microparticle and/or a nano-in-micro matrix) of the present technology includes chemiluminescence based sensing chemistry; for example, chemiluminescence based sensing chemistry that emits light in the presence of lactate, pyruvate, oxygen and/or hydrogen peroxide. In certain embodiments, a biosensor (for example a nanoparticle, a microparticle and/or a nano-in-micro matrix) may include a copolymer synthesized from one or more of: 4-hydroxyl benzyl alcohol; 1,8-octane diol; polyethylene imine; a fluorophore; or oxalyl chloride. In certain embodiments, the nanoparticles of a nano-in-micro matrix of the present technology are encapsulated in a polysaccharide based microparticle.

In some embodiments in which the biosensor is, or includes, a nanoparticle, microparticle and/or a nano-in-micro matrix, nanoparticles and/or microparticles are coated along with the polyelectrolyte coatings using layer by layer assembly technique over the encapsulated nanoparticles and/or microparticles. The polyelectrolyte in such embodiments may include one or more of PSS, PAH, PAA, chondroitin sulphate, collagen, dextran sulphate, PEI, chitosan, or alginate.

In certain aspects, a kit that includes a biosensor (for example a nanoparticle, a microparticle and/or a nano-in-micro matrix) as described herein is provided.

In some aspects, provided are methods for manufacturing a biosensor (for example a nanoparticle, a microparticle and/or a nano-in-micro matrix) as described herein.

In another aspect, provided is a method of measuring a biological analyte such as lactate, pyruvate, oxygen or hydrogen peroxide levels in a subject or sample.

The methods of the present technology may include implanting in the subject or adding to a sample a biosensor (for example a nanoparticle, a microparticle and/or a nano-in-micro matrix) such as described herein. The method may further involve using the biosensor (for example a nanoparticle, a microparticle and/or a nano-in-micro matrix) to detect and/or measure the analyte (such as lactate, pyruvate, oxygen or hydrogen peroxide) in the sample or subject. In certain embodiments the method involves implanting a biosensor (for example a nanoparticle, a microparticle and/or a nano-in-micro matrix) such as described herein into a subject in vivo. In some embodiments of the methods of the present technology, a biosensor (for example a nanoparticle, a microparticle and/or a nano-in-micro matrix) such as described herein is injected intramuscularly, intravenously, subcutaneously, intradermally or intraperitoneally in the subject. In certain embodiments of the methods described herein, the subject is one or more of: a mammal, a human, a rodent, a human patient, and/or an athlete.

In certain embodiments, the methods as described herein may include detecting and/or measuring light or chemiluminescence emitted from a biosensor; for example, light or chemiluminescence emitted by the biosensor in the presence of lactate, pyruvate, oxygen and/or hydrogen peroxide. In certain embodiments the method includes detecting and/or measuring fluorescence, luminescence, light, and/or chemiluminescence emitted from the biosensor when excited with light at 530 nm. In some embodiments, the method includes detecting and/or measuring fluorescence, luminescence, light, and/or chemiluminescence emitted from the biosensor in the near infra red (NIR) range (600 nm to 1100 nm). In some embodiments the method includes detecting and/or measuring fluorescence, luminescence, light, and/or chemiluminescence emitted from the biosensor at about 630 nm; or at about 650 nm; or at about 648 nm; or at about 653 nm. In some embodiments the method includes detecting and/or measuring fluorescence, luminescence, light, and/or chemiluminescence emitted from the biosensor at a wavelength above 600 nm; or above 625 nm; or above 645 nm; or at above 650 nm. In some embodiments the light or chemiluminescence is detected or measured using a luminometer, using a spectrophotometer, or using a confocal imaging or fluorescence imaging apparatus or technique. Accordingly, in certain embodiments a method as disclosed herein may include implanting in a subject or adding to a sample a biosensor (for example a nanoparticle, a microparticle and/or a nano-in-micro matrix) such as described herein, subsequently measuring light or chemiluminescence emitted from the biosensor, wherein the measured light or chemiluminescence indicates the level of an analyte such as lactate, pyruvate, oxygen or hydrogen peroxide in the subject or sample.

In certain aspects and embodiments of the methods as described herein, the method includes evaluating and/or monitoring the metabolic status of a subject by implanting a biosensor as described herein in the subject and using the biosensor to measure levels of lactate, pyruvate, oxygen and/or hydrogen peroxide. The levels of lactate, pyruvate, oxygen and/or hydrogen peroxide are indicative of metabolic status in the subject.

In some aspects and embodiments of the methods as described herein, the method includes measuring and/or monitoring ischemia in a subject by implanting a biosensor as described herein in the subject and using the biosensor to measure levels of lactate, pyruvate, oxygen and/or hydrogen peroxide. The levels of lactate, pyruvate, oxygen and/or hydrogen peroxide are indicative of ischemia in the subject.

In some aspects and embodiments of the methods as described herein, the method includes measuring lactate, pyruvate, oxygen and/or hydrogen peroxide in a food product. The method may include adding a biosensor as described herein in to the food product and using the biosensor to measure levels of lactate, pyruvate, oxygen and/or hydrogen peroxide. In certain embodiments the food product is one or more of: milk, koumiss, yogurt, kefir or cheese.

In certain embodiments of the methods described herein, the biosensor is used to measure oxygen and/or hydrogen peroxide and the amount of hydrogen peroxide and/or oxygen is used to calculate the amount of lactate in the subject or sample. In certain embodiments of the methods, the biosensor emits light or chemiluminescence in the presence of lactate, oxygen and/or hydrogen peroxide, and the light or chemiluminescence emitted from a biosensor in the subject or sample is measured.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 is a schematic representation of illustrative methods for preparation of micro and nanoparticles that may be used in producing biosensors of the present technology: (A) Emulsification technique for the preparation of alginate microspheres, (B) Syringe extrusion technique for alginate bead preparation, (C) Co-axial air-flow method of making alginate beads, (D) Mechanical disturbance to induce controlled breakup of droplets, (E) Electrostatic force applied to destabilize a liquid jet to make alginate microspheres, and (F) Electrostatic bead generator coupled with a tesla coil.

FIG. 2 is a diagram showing an exemplary method and system for generating nanoparticles, microparticles and nano-in-micro matrices of the present technology.

FIG. 3 shows schemes that may be used for lactate detection and measurement. Overall reaction of aryl-oxalate chemiluminescence

FIG. 4 shows an overall reaction of aryl-oxalate chemiluminescence

FIG. 5 is a schematic representation of an exemplary “smart tattoo” sensing concept in accordance with the present technology demonstrating exemplary implantation, interrogation, readout and components. Smart tattoo sensing is a concept where in sensing assay is implanted under the skin which is exposed to the interstitial fluid. The analytes present in the interstitial fluid are measured using analyte specific biological component like enzymes. The resulting reaction is measured by change in near infra red fluorescence due to NIR dyes. The excitation and emission of a NIR dyes can be measured using optical set up consisting of a source LED, optical fibres, and a fluorescence emission detecting system.

FIG. 6 (A, B, and C) show SEM images of illustrative polymeric nanoparticles (A), CaCO₃ micro/nanoparticles (B) and composite alginate microsphere (C) and TEM images of polymeric nanoparticles (D), CaCO₃ micro/nanoparticles (E).

FIG. 7 shows confocal images of (A) FITC-dextran loaded nanoparticles 150 KDa and (B) FITC-dextran loaded nanoparticles within alginate microparticles (nano-in-micro matrices).

FIG. 8 shows an illustrative fluorescence emission response of PtOEP in the presence of ambient conditions, and O₂ and CO₂ gas at an excitation of 530 nm.

FIG. 9 (a) shows illustrative effects of different gasses on fluorescence emission intensity of PtOEP normalized to changes of intensity on evaporation (All the gasses have been exposed for 10 sec using a pressure 90 mBar) and (b) illustrative effects of different gasses on fluorescence emission intensity of PtOEP. All the gasses have been exposed for 10 sec using a pressure 90 mBar.

FIG. 10 shows calibration curves for oxygen sensing in nanoparticles (--- [symbol] ---) and nano-in-micro matrix (--- [symbol] ----) using PtOEP dye encapsulated in PLA nanoparticles and PLA in alginate nano in micro system.

FIG. 11 shows dynamic response and reversibility of nano-in-micro matrix in response to O₂ concentration. Line represents the fluorescence intensity after exposure to oxygen or nitrogen and columns represent the corresponding O₂ concentration.

FIG. 12 shows an overlay of (a) oxygen sensing for nano-in-micro matrix and (b) overlay of lactate sensing using lactate oxidase encapsulated nano-in-micro matrix. Concentration of oxygen varies in from 0-15 mg/L.

### DETAILED DESCRIPTION

In the following detailed description, reference may be made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Unless otherwise stated, the singular forms “a,” “an,” and “the” as used herein include plural reference.

### Lactate and Detection of Lactate

The methods and biosensors of the present technology may, in certain embodiments, be used to detect and/or measure lactate, for example for the purpose of diagnosis and treatment of diseases, fitness and health monitoring in sports, in space medicine, in food applications in the food industry, in pharmaceutical applications and the pharmaceutical industry, for fermentation monitoring, and in polymer manufacturing applications.

Metabolic Status and Monitoring Thereof

In mammals, lactate is formed from the anaerobic metabolism of glucose in the glycolytic pathway during the process of fermentation of pyruvate catalyzed by lactate dehydrogenase (LDH). The concentration of blood lactate is usually 1-2 mmol/L at rest, but can rise to >20 mmol/L due to various conditions. Lactate levels do not increase in concentration until the rate of lactate production exceeds the rate of lactate metabolism. The production of excess lactic acid leads to production of acidic pH in the muscle proximity which causes the muscle fatigue. Lactate level alteration is commonly observed during power intensive exercise in healthy people and in some pathological conditions. Accordingly, in some embodiments, the methods and biosensors as described herein are used for lactic acid monitoring in mammals, for example athletes, sprinters and other exercising individuals and sports persons to monitor metabolic status, for example for the purpose of monitoring and/or estimating training status and fitness. During power-intensive exercises such as sprinting, when the rate of demand for energy is high, lactate is produced faster than the ability of the tissues to remove it and lactate concentration begins to rise. In some embodiments, the levels in lactate monitoring may be used to assist in optimizing training regimens, and or understanding the level of fitness achieved during training. The compositions and methods of the present technology be useful in various aspects of sports medicine, including rehabilitation from injury or for treatments of diseases that impact muscle including muscle wasting diseases, muscle degeneration, inability to control muscle and the like. A disturbance in the normal lactate level may be indicative of conditions of ischemia which may have arisen either due to shock, respiratory insufficiency, carbon monoxide intoxication, cyanide intoxication etc. or due to altered lactate metabolism which may have been caused due to diabetes or absorptive abnormalities of short chain fatty acids in the colon; accordingly the biosensors and/or methods described herein may be used to indicate, detect or measure such conditions. In certain embodiments of the methods and biosensors of the present technology, the analysis of lactate in saliva may be used as a preliminary diagnostic for cystic fibrosis. Several microorganisms responsible for tooth decay are responsible for production of D (+) lactic acid due to fermentation of carbohydrates in the food leading to dental cavities; accordingly in some embodiments the methods and biosensors disclosed herein may be used to measure or monitor lactic acid in a subject's mouth and/or teeth and in turn diagnose or identify tooth decay in the subject. Accordingly, in various embodiments the methods and biosensors as described herein may be used in the diagnosis and treatment of diseases, fitness and health monitoring in sports and space medicine.

Food and Fermentation Industry

Lactic acid is also known as “milk acid”, primarily found in sour milk products, such as: koumiss, yogurt, kefir and cheeses. Accordingly, in certain embodiments the methods and biosensors of the present technology may be used to detect and measure lactate in such food products. In illustrative embodiments, such methods are used to identify food products with an excess of lactic acid (for example to identify foods that may have spoiled or that are or are not suitable for human consumption). Tart flavor is produced by addition of lactic acid which also acts as a non volatile acidulant. As such the biosensors and methods may be used to monitor or evaluate the level of tart flavor in a food product. The casein in fermented milk is coagulated by lactic acid by using bacteria such as Bacillus acidilacti, Lactobacillus delbueckii or Lactobacillus bulgaricus to ferment carbohydrates from nondairy sources such as cornstarch, potatoes and molasses. Lactic acid has also found uses in various processed foods, as a pH adjusting ingredient, preservative and fermentation booster in rye and sourdough breads. Thus, the methods and biosensors of the present technology may be used to measure lactate in such food products during the manufacture process, for example, to optimize the amount of lactic acid added.

Polymer Industry

A variety of catalysts are used in conjunction to polymerize lactide to either heterotactic or syndiotactic polylactide or other copolymers. Polylactide is used as a biodegradable polymer with valuable drug delivery capabilities. Thus, in some embodiments the methods and biosensors of the present technology may be used to monitor, evaluate and/or optimize ingredients (lactate) in polymer manufacture.

Pharmaceutical Industry

Several antibiotics, drugs and chemicals employ and produce lactic acid as a substrate, catalyst or media. The ability to monitor and control such reactions can help in optimizing and increasing the yield of drugs like antibiotics which ultimately will cut down on manufacturing costs. Accordingly, in some embodiments the methods and biosensors of the present technology may be used to monitor, evaluate and/or optimize manufacture of pharmaceuticals or other chemicals.

### Biosensors

Biosensors of the present technology may be in any suitable form or made of any suitable media or matrix including, for example, without limitation, silicone rubbers, silica gels, polymers, PEG hydrogel and other sol-gels, alginate, CaCO₃ and may detect and/or measure analytes such as lactate, pyruvate, oxygen or hydrogen peroxide using any of the detection and measurement technologies described herein.

The term “biosensor” as used herein refers to any composition capable of detecting and/or measuring any biological analyte or biological process. In certain embodiments a biosensor includes one or more nanoparticles or a nanoparticle composition; in some embodiments a biosensor includes one or more nanoparticles or micropartcles, or a nanoparticle or microparticle composition; in some embodiments a biosensor includes a nano-in-micro matrix. In some embodiments nanoparticles of the present technology are between 1 nm and 2,000 nm and in various embodiments the microparticles of the present technology are between 600 nm and 100 μm.

Generation of Nanoparticles, Microparticles or a Nano-in-Micro Matrix

In certain embodiments the biosensor includes a nanoparticle, microparticle or a nano-in-micro matrix such as described herein. In some embodiments a screening assay or detection system as described herein is encapsulated within nanoparticles or microparticles; in some particular embodiments, a sensing assay system such as described herein is encapsulated in nanoparticles and the nanoparticles are incorporated into a nano-in-micro matrix.

The term “nanoparticle” or “nanosphere” as used herein refers to particles having a size (e.g., a diameter) between 1 nm and 2,000 nm; or between 1 nm and 600 nm; or between 50 nm and 500 nm; or between 100 nm and 400 nm; or between 150 nm and 350 nm; or between 200 nm and 300 nm, or between 500 and 700 nm. In certain embodiments, a “nanoparticle composition” as used herein refers to a composition that includes particles wherein at least 30%; or at least 40%; or at least 50%; or at least 60%; or at least 65%; or at least 70%; or at least 75%; or at least 80%; or at least 85%; or at least 87%; or at least 90%; or at least 92%; or at least 95%; or at least 97% of the particles fall within a specified size range, for example wherein the size range is between 1 and 2,000 nm; or between 1 nm and 600 nm; or between 50 nm and 500 nm; or between 100 nm and 400 nm; or between 150 nm and 350 nm; or between 200 nm and 300 nm. A nanoparticle as described herein may be made using any technique well known in the art and/or any of the methods described herein.

The term “microparticle” or “microsphere” as used herein refers to particles having a size (e.g., a diameter) between 600 nm and 100 μm; or between 650 nm and 7 μm; or between 650 nm and 5 μm; or between 700 nm and 4 μm; or between 750 nm and 3 μm; or between 800 nm and 2 μm. In certain embodiments, a “microparticle composition” as used herein refers to a composition that includes particles wherein at least 30%; or at least 40%; or at least 50%; or at least 60%; or at least 65%; or at least 70%; or at least 75%; or at least 80%; or at least 85%; or at least 87%; or at least 90%; or at least 92%; or at least 95%; or at least 97% of the particles fall within a specified size range, for example wherein the size range is between 600 nm and 100 μm; or between 650 nm and 7 μm; or between 650 nm and 5 μm; or between 700 nm and 4 μm; or between 750 nm and 3 μm; or between 800 nm and 2 μm. A microparticle as described herein may be made using any technique well known in the art and/or any of the methods described herein.

As used herein, a “nano-in-micro system,” “nano-in-micro matrix” or “nano-in-microparticle” refers to a microparticle that includes nanoparticles encapsulated within the microparticle. In some embodiments the size of the nanoparticles of the nano-in-micro matrix are between 1 nm and 600 nm and the size of the microparticles are between 650 nm and 5 μm.

Nanoparticles, microparticles, and/or nano-in-micro matrices of the present technology may be manufactured or generated using any method known in the art (for example see Kwok K K, et al. (1991) Production of 5-15 micron diameter alginate-polylysine microcapsules by an air atomization technique, Pharmaceutical Research 8: 340-44; Ribeiro C C, et al., (2004) Calcium phosphate-alginate microspheres as enzyme delivery matrices. Biomaterials 25: 4363-4373; Su J, Tao X, Xu H and Chen J F (2007) Facile encapsulation of nanoparticles in nanoorganized bio-polyelectrolyte microshells, Polymer 48: 7598-7603; and/or www.nanotechbriefs.com, Accessed on 26 Oct. 2008, each of which are hereby incorporated by reference in their entireties); in some embodiments nanoparticles, microparticles, and/or nano-in-micro matrices my be generated using emulsification techniques, syringe extrusion techniques, coaxial air flow method, mechanical disturbance method, electrostatic method. In certain embodiments nanoparticles, microparticles, and/or nano-in-micro matrices of the present technology are made using methods similar to those disclosed in Lee et al. ((2007) In vivo imaging of hydrogen peroxide with chemiluminescent nanoparticles, Nature letters 1-5).

In certain embodiments, a nano-in micro matrix is used as a biosensor in the present technology. In some embodiments the nano-in micro matrix may have certain advantages over other biosensors including nanoparticles and microparticles. For example, in embodiments in which biosensors are administered in vivo, a nano-in-micro matrix may be resistant to uptake of the biosensor by the animal's reticulo-endothelial system which may, in turn, prevent an immune response that can often occur with other biosensors such as nanoparticles. Such immune response does not always occur, or the immune response may not cause significant side effects, thus in certain embodiments the biosensor is, or includes, nanoparticles that are not part of a nano-in-micro matrix.

Emulsification Technique

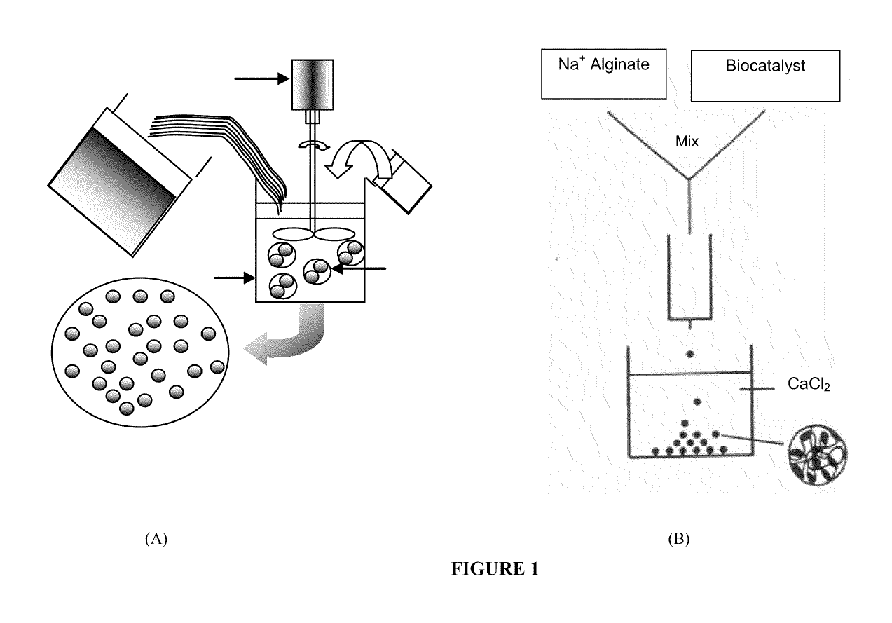

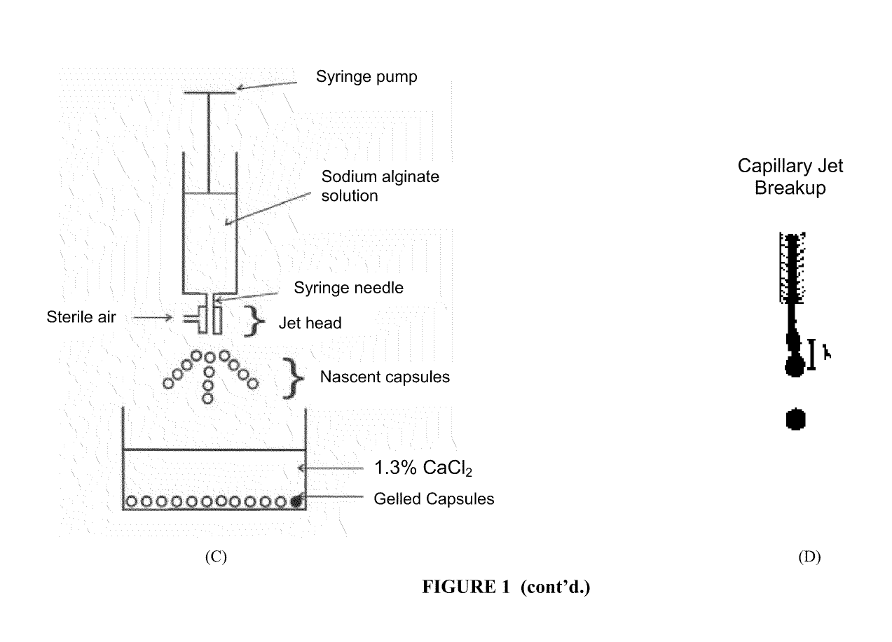

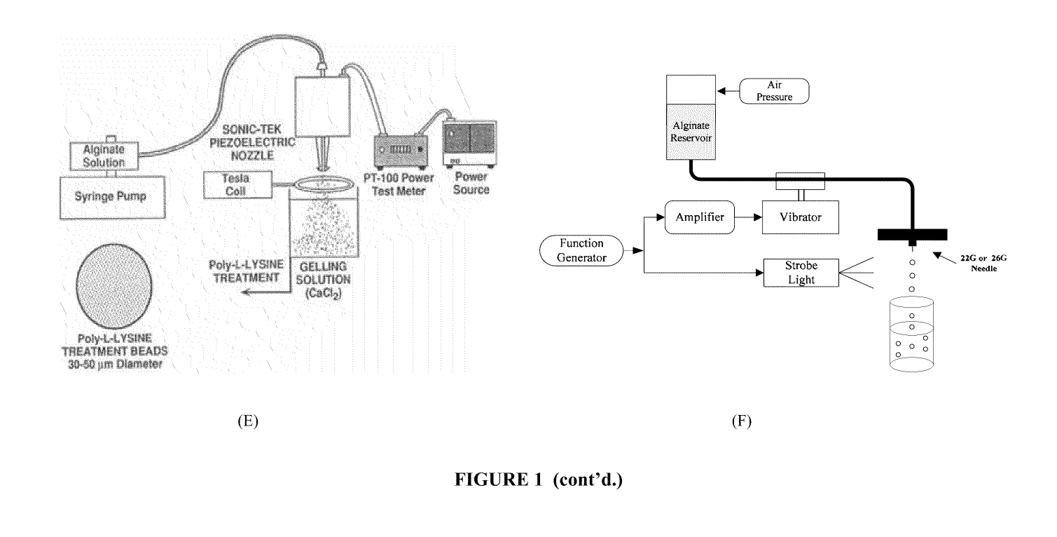

In this method of generating nanoparticles, microparticles, and/or nano-in-micro matrices in accordance with the present technology, the aqueous solution may be mixed in an non-aqueous phase containing an emulsifier to form emulsion droplets (FIG. 1A). The solution may then be gelled with a gelling agent. Crosslinking may be optionally done and the crosslinked emulsion may subsequently be inverted in presence of excess water to form aqueous dispersion of uniform sized nanoparticles and/or microparticles. The process parameters such as stirring speed, rate of addition of gelling agent and concentration and composition of encapsulating polymer can be altered to affect the size, shape and surface characteristics of the microparticles and or nanoparticles.

Syringe Extrusion Technique

In this method of generating nanoparticles, microparticles, and/or nano-in-micro matrices in accordance with the present technology, beads (often larger than 1 mm diameter) are produced and may be prepared by using a syringe with a needle or pipette (see FIG. 1B). Encapsulant-containing solution may be added in a drop wise manner in the crosslinking solution. The particle size may be altered by changing the syringe dimensions.

Coaxial Air Flow Method

This method of generating nanoparticles, microparticles, and/or nano-in-micro matrices in accordance with the present technology, may involve the use of a concentric stream of air which shears the liquid droplets released from the needle. The size of particles generated is controlled by the air flow velocity, viscosity of encapsulant and the distance of the needle to the solution of crosslinking agent (FIG. 1C).

Mechanical Disturbance Method

In this method of generating nanoparticles, microparticles, and/or nano-in-micro matrices in accordance with the present technology, liquid droplets are broken into fine droplets using a mechanical disturbance. In some embodiments, vibrations are used for producing the mechanical disturbance. In various embodiments, this method may produce particles of the order of 500 nm-2 mm with a corresponding flow range of about 5-500 ml/min (FIG. 1D).

Electrostatic Force Method

In this method of generating nanoparticles, microparticles, and/or nano-in-micro matrices in accordance with the present technology, electrostatic forces are used to destabilize a viscous jet, where the electrostatic force is used to disrupt the liquid surface instead of a mechanical disturbance (see for example FIG. 1F). In some embodiments, the electrostatic method produces beads in the 0.05-5 mm range.

Electrostatic Bead Generator Method

In this method of generating nanoparticles, microparticles, and/or nano-in-micro matrices in accordance with the present technology, a piezoelectric nozzle is used for forming droplets uses electrostatic charging of droplets as they fall in a crosslinking solution. Surfactants may also be used for formation of small droplets in the solution. Another feature of this method is that it may use a tesla coil to impart necessary charge on the droplets. This method is reported for the coating of poly-L-lysine solution (FIG. 1F).

Use of Droplet Generators

In this method of generating nanoparticles, microparticles, and/or nano-in-micro matrices in accordance with the present technology, droplet generators are useful for producing polysaccharide microparticles (optionally containing nanoparticles to make a nano-in-micro matrix) which are either based on electrostatic field or air driven mechanism. The electrostatic mechanism may utilize a potential difference between a capillary tip such as a nozzle and a flat counter electrode reduce the diameter of droplets by applying an additional force (i.e. electric force) in the direction of gravitational force in order to overcome the upward capillary force of liquid. These can be used to produce small droplets <100 μm and such droplets may be produced from highly viscous liquids depending on their conductivity. Electrostatic droplet generation is an effective technique for producing extremely small uniform microbeads less then 150 μm diameters. A method was developed for animal cell immobilization based on calcium alginate/PLL coated microbeads produced by electrostatic spraying for culturing surface immobilization of insect cells. Air driven droplet generator have also been used for a similar purpose by Kwok et al. for immobilization of Bacillus Calmette Guerin (BCG) vaccine. In some embodiments, an air-driven multi-needle droplet generators which is used for grafting alginate-poly-L-lysine encapsulated islets. The size of the droplets can be modulated depending on the diameter of the jacket, the air flow rate, and the outer diameter of the nozzle, whereas the production rate depends upon the pressure on the alginate, and on the diameter and the length of the nozzle.

Modified Method

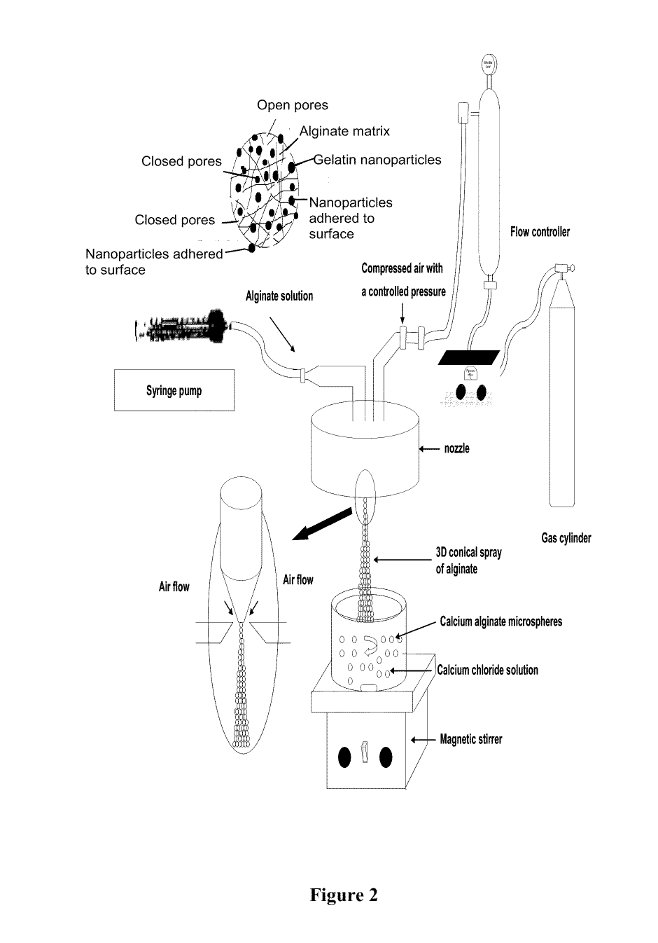

In certain embodiments, nanoparticles, microparticles, and/or nano-in-micro matrices in accordance with the present technology are made in accordance with the procedures of Lee et al. ((2007) In vivo imaging of hydrogen peroxide with chemiluminescent nanoparticles. Nature letters 1-5) or modifications thereof. In some embodiments, the methods of Lee et al. are adapted/modified such as to allow for manufacture of nano-in micro matrices. In certain embodiments, the nanoparticles, microparticles, and/or nano-in-micro matrices are made using the processes shown on FIG. 2.

In certain preferred embodiments a nano-in-micro matrix may be made using general procedures as follows. Polymeric nanoparticles may first be prepared using a copolymer of oxalyl chloride, 4-hydroxybenzyl alcohol, 1,8-octane diol and polyethylene imine by the emulsification method or CaCO₃ micro/nanoparticles may be prepared using precipitation method, by mixing two counter-ions Na₂CO₃ and CaCl₂ in equimolar concentration. The nanoparticles may include components for analyte detection, sensing and/or measurement such as described herein. Sodium alginate microparticles having the nanoparticles contained therein may then be prepared using a droplet generator (see for example FIG. 2), for which the parameters like flow rate of the sodium alginate solution, concentration of sodium alginate, concentration of calcium chloride, distance of the nozzle from the surface of liquid, air pressure, may be optimized in order to get a desired size range; for example, in certain embodiments the microparticles are less than 10 μm.

The microspheres or the nanoparticles may be characterized using SEM and TEM. In certain embodiments polymeric nanoparticles are in the size range of 200-300 nm and the CaCO₃ microparticles are between 800 nm-2 micron; however the nano-in-micro matrix can be prepared of different size ranges depending on instrument parameter optimization.

In certain embodiments, ruthenium based dye and/or porphyrin can be encapsulated in the micro or nanoparticles as an oxygen sensing component; for example by mixing the ruthenium based dye in sodium alginate and then forming nanoparticles, microparticles, or nano-in-micro matrices using optimized parameters in a droplet generator such as described herein. Visual detection of the fluorescence, luminescence, light, and/or chemiluminescence from such micro or nanoparticles, microparticles, or nano-in-micro matrices can be performed, for example by using confocal fluorescence imaging techniques, fluorescence microscopic techniques; fluorimeters, spectrophotometers, luminometers, or any other suitable imaging device or system.

In some embodiments a lactic acid catalyzing enzyme (for example, lactate oxidase (Lox), lactate dehydrogenase, and/or lactate monooxygenase) may be immobilized in alginate nanoparticles and/or microparticles or the nanoparticles and/or microparticles will be coated along with the polyelectrolyte coatings using layer by layer assembly technique over the encapsulated nanoparticles nanoparticles and/or microparticles. The polyelectrolyte in such embodiments may include one or more of PSS, PAH, PAA, chondroitin sulphate, collagen, dextran sulphate, PEI, chitosan, or alginate. When the enzyme (e.g., lactate oxidase) is encapsulated in the core, a dye (such as any fluorophore or any near infra red fluorophoure e.g. Alexa Flour) may be chemically attached to the enzyme such as to facilitate visualization. In certain embodiments, enzyme containing nanoparticles, microparticles and nan-in-micro matrices are generated using the methods disclosed in Abhijeet B. Joshi and Rohit Srivastava; Polyelectrolyte Coated Calcium Carbonate Micro-particles as Templates for Enzyme Encapsulation Advanced Science Letters Vol. 2, 1-8, 2009 (1 Sep. 2009) (hereby incorporated by reference in its entirety) or variations of such methods.

### Detection Systems, Methods and Compositions

Various aspects and embodiments of the compositions and methods of the present technology involve detection and or measurement of analytes. Such detection and measurement may include or involve technologies, principles, compositions and/or methods as described below for detection of lactate, pyruvate, hydrogen peroxide and/or oxygen. For example, detection components as described below may be incorporated into a biosensor as described herein. In some embodiments, the components of the detection system are included within and/or on the surface of nanoparticles, microparticles and/or nano-in-micro matrices. In certain embodiments, the components of one or more detection system are incorporated within nanoparticles that are part of a nano-in-micro matrix.

Detection and Measurement of Lactate

In many embodiments of the methods and biosensors of the present technology, the basis of detecting and measuring lactate may involve the metabolism of lactate to form pyruvate which is accompanied by consumption of oxygen and production of hydrogen peroxide.

L-lactate+O₂→Pyruvate+H₂O₂

In this regard, the detection or measurement of lactate in the present technology may involve or include one or both of detecting and or measuring hydrogen peroxide or oxygen in a sample or subject. The levels of hydrogen peroxide and/or oxygen may, in turn, be used to calculate or extrapolate the levels of lactate.

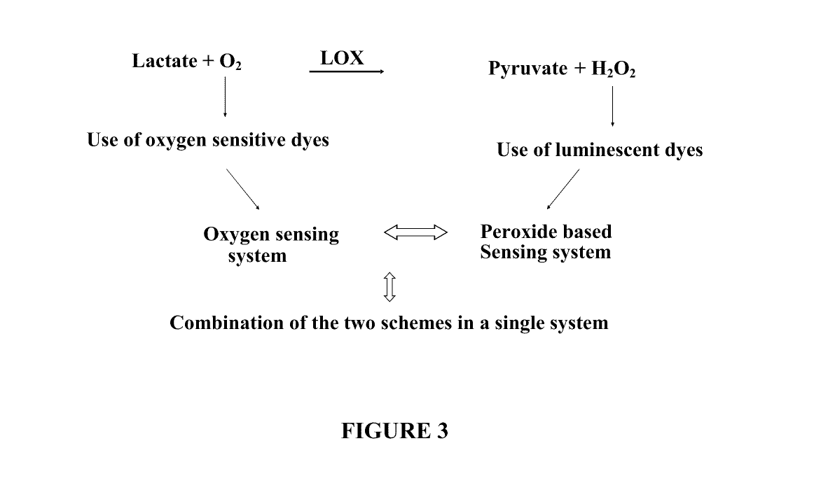

In certain embodiments detection and measurement of lactate involves schemes such as outlined in FIG. 3.

Hydrogen Peroxide Detection and Measurement

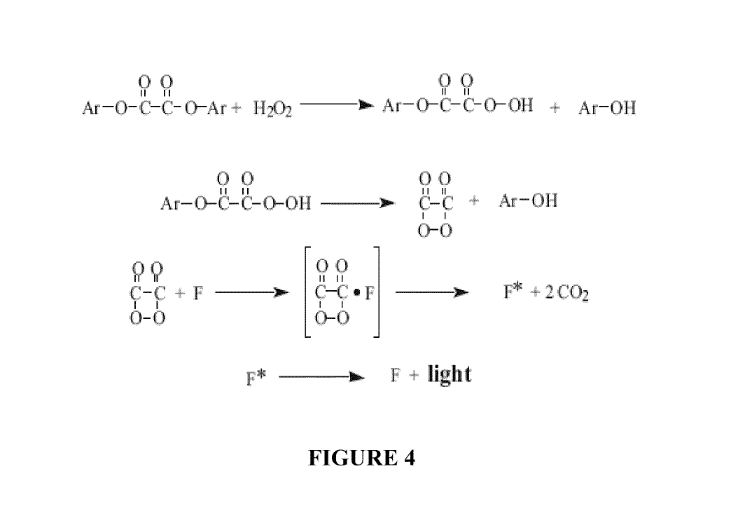

In certain embodiments of the methods and biosensors of the present technology, hydrogen peroxide is detected. Such detection may utilize a peroxyoxalate chemiluminescence system. In this regard some esters of oxalic acid, mainly aryl-oxalates, react with the oxidant hydrogen peroxide in the presence of suitable fluorescers to give rise to light emission. The chemical system is may be referred to as a “peroxyoxalate chemiluminescence system” and is an energy transfer system. By reacting with hydrogen peroxide, the oxalic acid ester gives an unstable intermediate of high energy, 1,2-dioxetanedione, which in turn excites the fluorescer. The excited fluorescer returns to the ground state emitting light in a typical fluorescence process. The overall reaction of aryl-oxalate chemiluminescence is shown in FIG. 4. The production of 1,2-dioxetanedione proceeds in two steps with first, the production of one phenol molecule and peroxyoxalic acid which decomposes into a second phenol molecule and 1,2-dioxetanedione intermediate. Then, the intermediate forms a charge-transfer-type complex with the fluorescer. This unstable intermediate releases excited fluorescer and two carbon dioxide molecules. In an illustrative embodiment, one exemplary oxalic acid that can be used in a peroxyoxalate chemiluminescence system of the present technology is TCPO (bis-(2,4,6-trichlorophenyl)oxalate). With suitable fluorescers such as perylene, rubrene or 9,10-diphenylanthracene the quantum yield of the chemiluminescence reaction may be in the order of 0.22-0.27 and the color of the emitted light depends on the fluorescer used. The chemiluminescence reaction of esters of oxalic acid can proceed within a wider pH range than for luminol.

Accordingly, in certain embodiments of the methods and biosensors of the present technology that involve or include detection of hydrogen peroxide, peroxalate esters are used for the determination of hydrogen peroxide. In such embodiments peroxalate esters may be converted to a dioxetanedione intermediate which is a high energy compound, with capability to excite fluorophores. In the presence of H₂O₂ a peroxalate moiety is converted to dioxetanedione, which subsequently excites pentacene giving an emission at the wavelength of 630 nm.

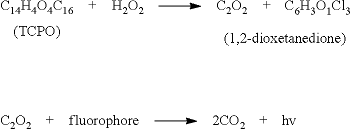

In some embodiments, a pentacene dye may be encapsulated in peroxalate nanoparticles; for example as described in Lee et al. ((2007) In vivo imaging of hydrogen peroxide with chemiluminescent nanoparticles. Nature letters 1-5) which is hereby incorporated by reference in its entirety. In certain embodiments, pentacene dye/peroxalate nanoparticles, for example such as described in Lee et al. may be incorporated in a nano-in-micro matrix such as described herein.

Oxygen Detection and Measurement

In certain embodiments, the methods and biosensors of the present technology are used to detect and/or measure oxygen levels in a sample or subject. The detection of oxygen may involve the use oxygen sensitive dyes (e.g., near infra red dyes) for the determination of depletion of oxygen, for example, ruthenium based dyes and/or porphyrins may be used for detection and measurement of oxygen. In certain embodiments oxygen detecting dyes and detection systems such as described in the following references (each hereby incorporated by reference in their entireties): Araki N, et al. (2007) Optical oxygen-sensing properties of porphyrin derivatives anchored on ordered porous aluminium oxide plates. Photochemical & photobiological sciences: Official journal of the European Photochemistry Association and the European Society for Photobiology 6: 794-803; Basu B J (2007) Optical oxygen sensing based on luminescence quenching of platinum porphyrin dyes doped in ormosil coatings. Sensors & Actuators: B. Chemical 123: 568-577; Oter O, et al. (2008) Photocharacterization of novel ruthenium dyes and their utilities as oxygen sensing materials in presence of perfluorochemicals. Journal of fluorescence 18: 269-276 may be used in the methods and biosensors of the present technology.

Oxygen sensitive fluorescent dyes that may be utilized in the present technology include ruthenium based dyes (tris(1,10-phenanthroline)ruthenium(II) [Ru (phen)₃], tris(4,7-diphenyl-1,10-phenanthroline)ruthenium(II) chloride [Ru (dpp)₃], and tris(5-acrylamido-1,10-phenanthroline)ruthenium(II)), Pt and Pd porphyrins and decacyclene. In certain embodiments ruthenium based dyes are used due to relative chemical and photostability. Such dyes may be used in different matrices including silicone rubbers, silica gels, polymers, PEG hydrogel and other sol-gels.

In some embodiments porphyrins are present in the CaCO₃ matrix of a biosensor (such as a nanoparticle, microparticle or nano-in-micro matrix) and/or encapsulated within a nanoparticle, microparticle or nano-in-micro matrix. In some embodiments, the incorporation of oxygen sensitive dyes in the biosensor will provide a ratiometric analysis of the intensities of fluorescence of the oxygen sensitive dye. In some embodiments the biosensor further includes a oxygen insensitive dye; and in certain embodiments the fluorescence of the oxygen sensitive dye is compared to that of the oxygen insensitive dye.

Detection and Measurement of Fluorescence and/or Chemiluminescence

In various aspects and embodiments, the methods and biosensors of the present technology utilize enzyme based sensors which employ fluorescence property of materials, preferably serving the purpose of increasing one or the other or both the specificity and sensitivity of the detection and/or measurement of a metabolite in a matrix. Certain embodiments of such fluorescence-based methods and biosensors may involve quenching behavior governed by the Stern-Volmer equation:

$$\frac{{I}_{0}}{I} = 1 + {κ}_{sv} [ Q ]$$

where I₀ and I are the fluorescent intensities in the absence and the presence of the quencher, Q is the concentration of the quencher, Ksv is the Stern-Volmer quenching constant.

Detection and/or measurement of the fluorescence, luminescence, light, and/or chemiluminescence from biosensors of the present technology (such nanoparticles, microparticles, or nano-in-micro matrices) can be performed, for example by using confocal fluorescence imaging techniques, fluorescence microscopic techniques; fluorimeters, spectrophotometers, luminometers, or any other suitable imaging device or system. In some embodiments detection and/or measurement of the fluorescence, luminescence, light, and/or chemiluminescence from biosensors of the present technology (such nanoparticles, microparticles, or nano-in-micro matrices) can be performed, using an imaging system such as an IVIS imaging system (Xenogen, USA).

Administration or Application of Biosensors to Subjects or Samples and Measurement of Analytes.

In some embodiments a biosensor (for example a nanoparticle, a microparticle and/or a nano-in-micro matrix) such as described herein is added to a sample for detection or measurement of an analyte. For example in applications used to detect or measure lactate, pyruvate, hydrogen peroxide and or oxygen in food products or in a fermentation process, a biosensor (for example a nanoparticle, a microparticle and/or a nano-in-micro matrix) may be added or mixed into a sample of food or fermentation product in an amount suitable for proper detection of the desired analyte. Likewise measurements taken for analyzing ingredients such as lactate in the polymer and pharmaceutical industry may involve adding or mixing a biosensor of the present technology into an appropriate sample for those applications. In some embodiments, a method of detecting and/or measuring an analyte such as described herein in a sample includes adding a biosensor of the present technology to the sample; measuring light, fluorescence and/or chemiluminescence emitted from the biosensor; and using the amount of light, fluorescence and/or chemiluminescence emitted from the biosensor to indicated the presence or absence of the analyte in the sample and/or to calculate the amount of the analyte in the sample. In certain embodiments of the method, the biosensor may be left in the sample to incubate for a specified amount of time before the light, fluorescence and/or chemiluminescence emitted from the biosensor is measured; for example the amount of time may be about one minute, two minutes; four minutes; 8 minutes; 15 minutes; 30 minutes; 45 minutes; 1 hour; two hours or more. In some preferred embodiments the method is used for continuous monitoring of the amount of analyte in the sample; that is the biosensor may be added to the sample and more than one (for example, at least 2; 3; 4; 5; 6; 7; 8; 9; 10; 15; 25; 50; 100 or more) measurements may be taken and the presence and/or amount of the analyte may be tracked over time. In certain embodiments the presence and/or amount of the analyte may be monitored in accordance with the present technology in “real time,” i.e., continuous monitoring throughout process, manufacturing step or steps, or procedure. In some embodiments the methods and/or biosensors of the present technology may include the use of one or more controls; for example a reference sample or mechanism to correct for background or to create a standard curve. In some embodiments of biosensors sensitive or responsive to a particular analyte, the control includes a reference dye that is not sensitive to that analyte.

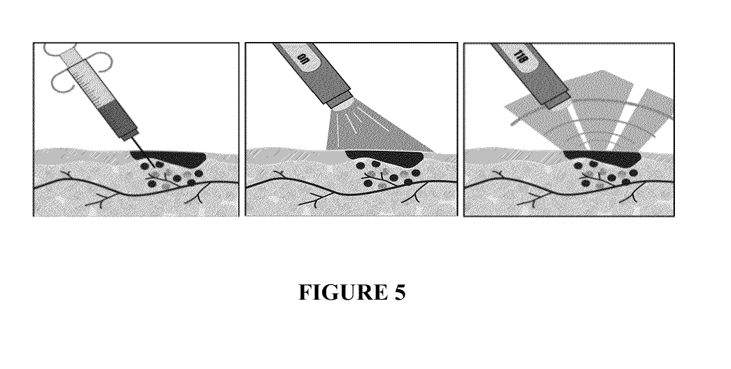

In some aspects and embodiments a biosensor (for example a nanoparticle, a microparticle and/or a nano-in-micro matrix) of the present technology may be implanted, inserted or injected into a subject in vivo; for example in applications for measuring an analyte (such as lactate, pyruvate, hydrogen peroxide and/or oxygen) for purposes such as diagnosis and treatment of diseases, fitness and health monitoring, space medicine, monitoring metabolic status; monitoring an inflammatory response; measuring ischemia and the like). In some illustrative embodiments, the smart tattoo concept such as shown on FIG. 5 may be used in the present technology. In various embodiments, a biosensor may be injected or inserted in to the subject subcutaneously, intramuscularly, intravenously, subcutaneously, intradermally, and/or intraperitoneally. In some embodiments the biosensor is injected or inserted into a deep tissue of the subject. In certain embodiments a biosensor is injected or inserted into the muscle of a subject, for example muscle in the leg, arm, or abdomen of the subject; such embodiments may be advantageous in methods for monitoring fitness and health, monitoring space medicine, monitoring metabolic status; and measuring ischemia. In some embodiments the biosensor is injected intramuscularly at a depth of between about 0.5 mm and about 10 mm; for example at about 1 mm; 2 mm; 3 mm; 4 mm; 5 mm; 6 mm; 7 mm; 8 mm; 9 mm; or 10 mm. In some embodiments the biosensor is injected into the peritoneum of the subject; such embodiments may be advantageous in methods for monitoring fitness and health, monitoring space medicine, monitoring metabolic status; measuring ischemia as well as for methods of monitoring or diagnosing inflammatory diseases such as arthrosclerosis, chronic obstructive pulmonary disease and liver hepatitis. In certain embodiments of the method, the biosensor may be left in the subject to incubate for a specified amount of time before the light, fluorescence and/or chemiluminescence emitted from the biosensor is measured; for example the amount of time may be about one minute, two minutes; four minutes; 8 minutes; 15 minutes; 30 minutes; 45 minutes; 1 hour; two hours or more. In some preferred embodiments the method is used for continuous monitoring of the amount of analyte in the subject; that is the biosensor may be added to the sample and more than one (for example, at least 2; 3; 4; 5; 6; 7; 8; 9; 10; 15; 25; 50; 100 or more) measurements may be taken and the presence and/or amount of the analyte may be tracked over time. In certain embodiments the presence and/or amount of the analyte may be monitored in accordance with the present technology in “real time,” i.e., continuous monitoring throughout an exercise regimen; a specified period of time, a surgery, an activity, a test, a procedure, an event such as a cardiac emergency, etc. In some embodiments the methods and/or biosensors of the present technology may include the use of one or more controls; for example a reference sample or mechanism to correct for background or to create a standard curve. In some embodiments of biosensors sensitive or responsive to a particular analyte, the control includes a reference dye that is not sensitive to that analyte.

Kits

The biosensors (for example a nanoparticle, a microparticle and/or a nano-in-micro matrix), materials and components described herein may be suited for the preparation of a kit. Thus, the disclosure provides a detection kit useful for determining the presence and or amount of an analyte such as, for example, lactate, pyruvate, oxygen and/or hydrogen peroxide.

In one embodiment, the methods described herein may be performed by utilizing pre-packaged diagnostic kits including materials (such as a biosensor, a nanoparticle, a microparticle and/or a nano-in-micro matrix) and/or reagents to perform any of the methods of the technology. The kits may contain instructions for the use of the reagents and interpreting the results. In some embodiments, each component of the kit can be enclosed within an individual container and all of the various containers can be within a single package, along with instructions for interpreting the results of the assays performed using the kit.

### EXAMPLES

The present compositions, methods and kits, thus generally described, will be understood more readily by reference to the following examples, which are provided by way of illustration and are not intended to be limiting of the present methods and kits. The following is a description of the materials and experimental procedures used in the Examples.

### Example 1

### Preparation of Peroxalate Nanoparticles and Dye Loaded Peroxalate Nanoparticles

### Synthesis of Peroxalate Polymer

Peroxalate polymer was synthesized according to a modified method developed by Lee et al. Briefly, 4-Hydroxybenzyl alcohol (16 mmol) and 1,8-octanediol (2.4 mmol) were dissolved in dry tetrahydrofuran (10 ml), to which triethylamine (40 mmol) was added dropwise at 0° C. This mixture was added to oxalyl chloride (18.3 mmol) in dry tetrahydrofuran (20 ml) at 0° C. The reaction was kept at room temperature overnight.

### Purification of Polymer

The reaction mixture was purified by quenching with a saturated brine solution, and extracted with ethylacetate. The combined organic layers were dried over anhydrous Na₂SO₄ and concentrated under vacuum. The polymer was isolated by precipitating in dichloromethane/hexane (1:1).

### Preparation of Peroxalate Nanoparticles

Polymer (35 mg) was dissolved in 8 ml of dichloromethane, the mixture was added to 8 ml of a polyvinyl alcohol solution (5.0% in phosphate buffer pH 7.4) to form a fine oil/water emulsion. A nanoparticle suspension was prepared by rotary evaporation of dichloromethane for 30 min.

### Encapsulation of Fluorophore in Nanoparticles

A fluorophore (1 mg/ml) is mixed with polymer solution in THF, and nanoparticles are formed as in the previous section. The dye loaded nanoparticles are then separated by centrifugation at 21000 g for 15 min. The dye loaded nanoparticles were characterized by TEM and a fluorescence spectrophotometer.

### Example 2

### Preparation of PtOEP Loaded PLA Nanoparticles

PtOEP solution in acetone (0.02 mg/ml) was mixed with PLA so that the dye concentration is 1% w/w of polymer. The PtOEP and PLA solution in acetone was then added to a 1% w/w PVA surfactant solution. The mixture was stirred overnight for formation of nanoparticles. The residual solvent was evaporated using rotavapour at 40° C. The formed nanoparticle suspension was filtered to remove precipitated dye aggregates. The filtered suspension was washed using deionized water thrice to remove adhering surfactant.

### Example 3

### Preparation of PtOEP Loaded CaCO₃Nanoparticles

PtOEP was encapsulated using a modified emulsification method similar to that of Example 1, wherein the PtOEP was dissolved in DCM (1 mg/ml) and added to a preformed suspension of PSS doped CaCO₃ microparticles. The reaction mixture was stirred vigorously using a cyclomixer and DCM was allowed to evaporate at ambient conditions. The evaporation of DCM led to transition of dye into the CaCO₃ microparticles. The dye loaded microparticles were washed three times using centrifugation at 15000 g for 10 min by double distilled water. The dye encapsulated microparticles were characterized using fluorescence spectrophotometer and fluorescence microscope. PtOEP was encapsulated in CaCO₃ microparticles using a modified emulsification method such as in Example 1, in which PtOEP due to its lipophilic nature makes a transition into the inorganic microparticle phase. This occurs due to reduced surface energy in the oil droplets containing the dye when present with the inorganic phase, rather than the aqueous phase. Another important factor which may govern the encapsulation of dye in the microparticles in some embodiments is the surface tension of the organic phase. In such embodiments, the organic phase may show reduced surface tension presence of the inorganic phase than when present only in an aqueous phase.

### Example 4

### Entrapment of Nanoparticles in Alginate Microspheres (Nano-in-Micro Particles)

Sodium alginate microparticles having the above nanoparticles contained therein are then prepared using a droplet generator consistent with the FIG. 2 diagram. The parameters such as flow rate of the sodium alginate solution, concentration of sodium alginate, concentration of calcium chloride, distance of the nozzle from the surface of liquid, air pressure, may be optimized in order to get a desired size range; for example, in certain embodiments the microparticles are less than 10 μm.

### Example 5

### Characterization of Nanoparticles, Microparticles and Nano-in-Micro Particles

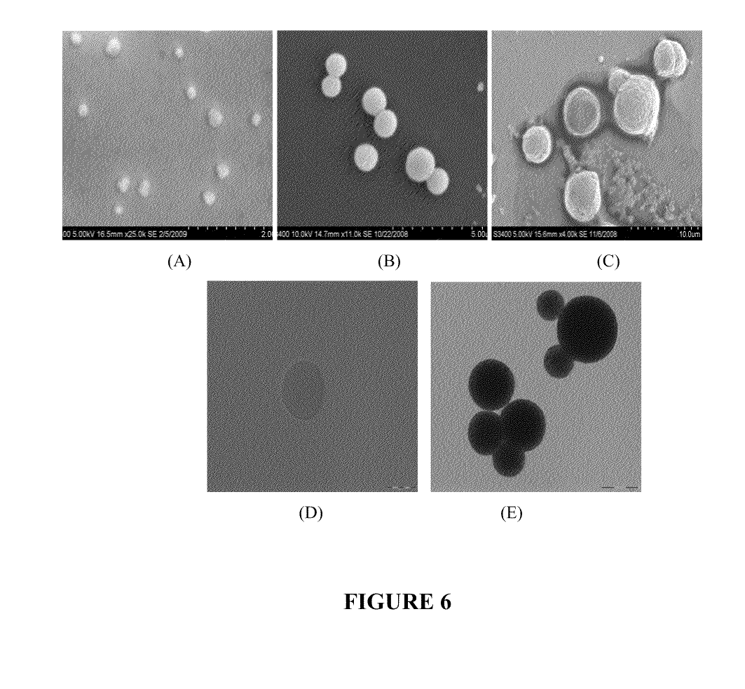

Nanoparticles and nano-in-micro particles were made according to the general procedures of Example 1 and were characterized using DLS, SEM and TEM. PLA nanoparticles were found to be in the size range of 200-300 nm, dye loaded PLA nanoparticles show a size of 500 nm, dye loaded peroxalate nanoparticles showed a size of 430 nm with a polydispersity of 0.25 and that of CaCO₃ nanoparticles 800 nm-2 micron (see FIG. 6). The size of nano in micro alginate matrix was 5μ±3μ.

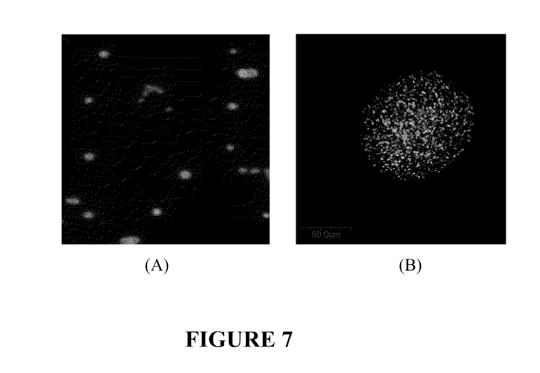

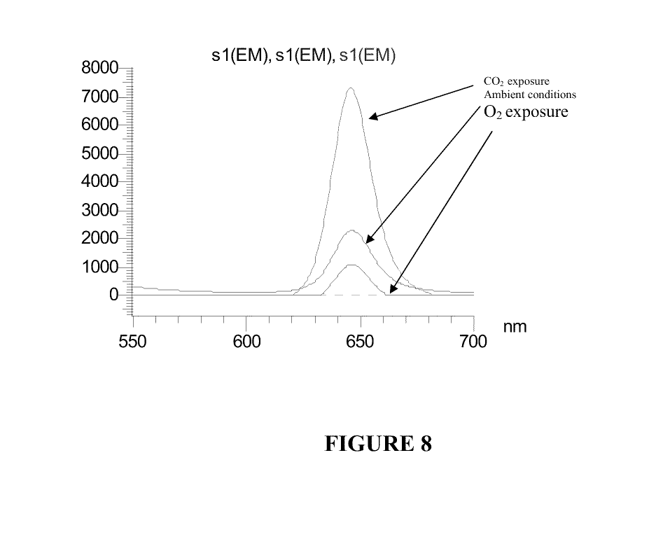

FITC-dextran was loaded as a model compound in nanoparticles, which were then loaded in alginate microparticles (nano-in-micro matrices) as described in the above Examples. The FITC-dextran loaded nano-in-micro matrices were characterized using confocal fluorescence imaging (FIG. 7).

### Example 6

### Biosensors Having Lactate Oxidase

2 mg/ml of lactate oxidase was premixed with alginate solution and then dye loaded nanoparticles made in accordance with Example 1 were mixed to form a suspension. The suspension was sprayed through a droplet generator to form nanoparticle encapsulated microparticles (nano-in-micro matrix) consistent with the Example 1 procedures and the nano-in-micro matrix is used for sensing.

### Example 7

### Oxygen Sensing Biosensors

### Oxygen Sensing in PLA Nanoparticles, Nano in Micro Matrix and CaCO₃ Nanoparticles

500 μl of PtOEP encapsulated nanoparticles/microparticle suspension was re-suspended in water to make 2 ml of suspension. The suspension was then exposed to different gasses. The exposure of dye loaded particle suspension to different gasses like atmospheric air, Nitrogen and Oxygen was followed by capture of fluorescence emission scan. Different concentration of O₂ was determined using an Orion DO meter. The gasses were bubbled in the suspension and allowed to stir for 10 seconds and 20 seconds, and then analyzed using fluorescence spectro-photometry.

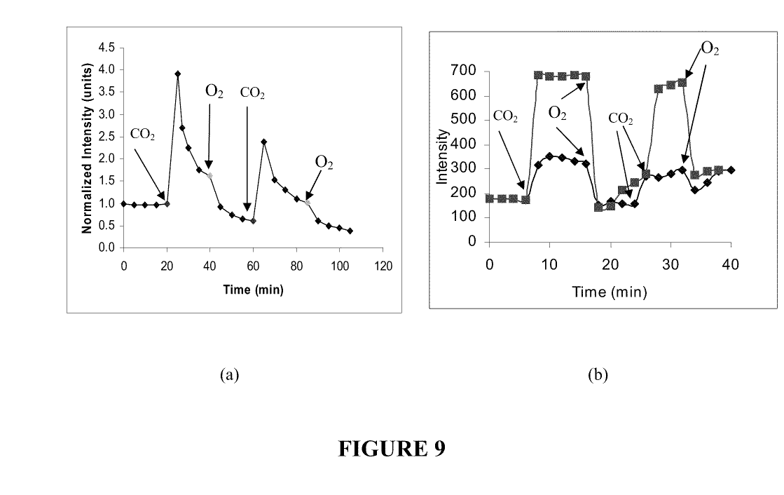

FIG. 9 (a) indicates that fluorescence emission intensity increases to almost 4 times in response to CO2 bubbled for 10 seconds. The fluorescence emission intensity in response to O₂ shows a quenching effect up to 2 times. Similar effect was also seen in the second cycle of exposures. However the peak intensities achieved after exposure was reduced.

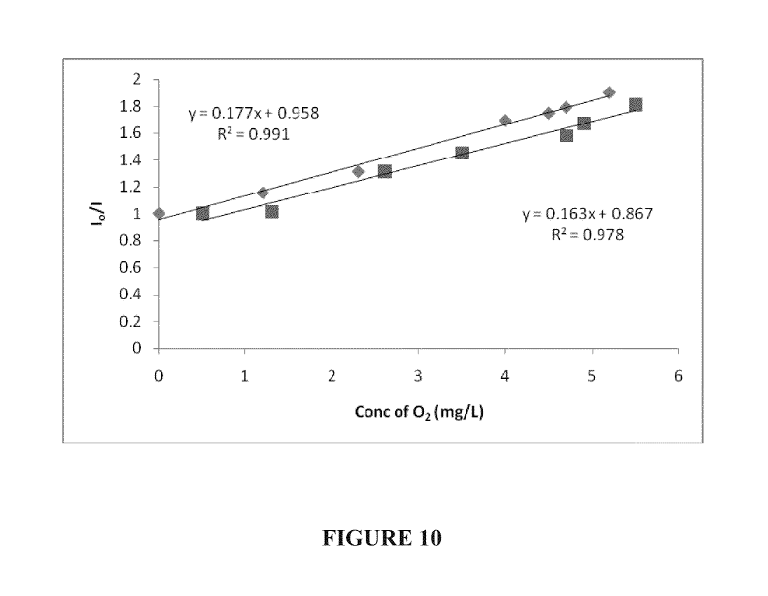

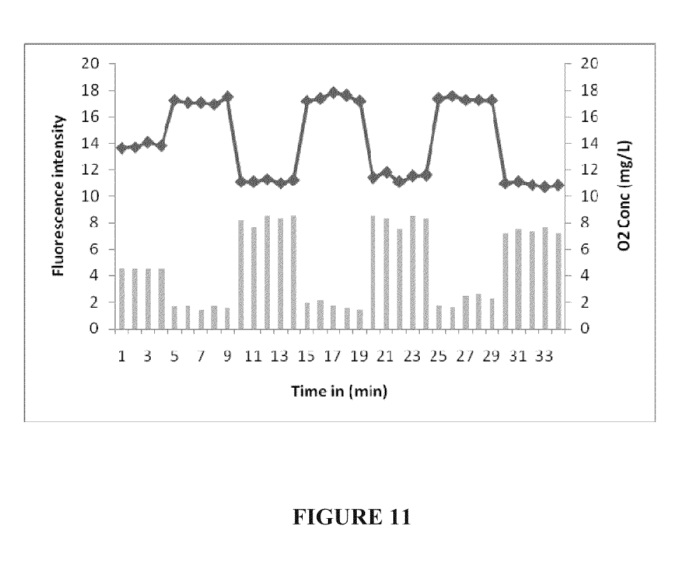

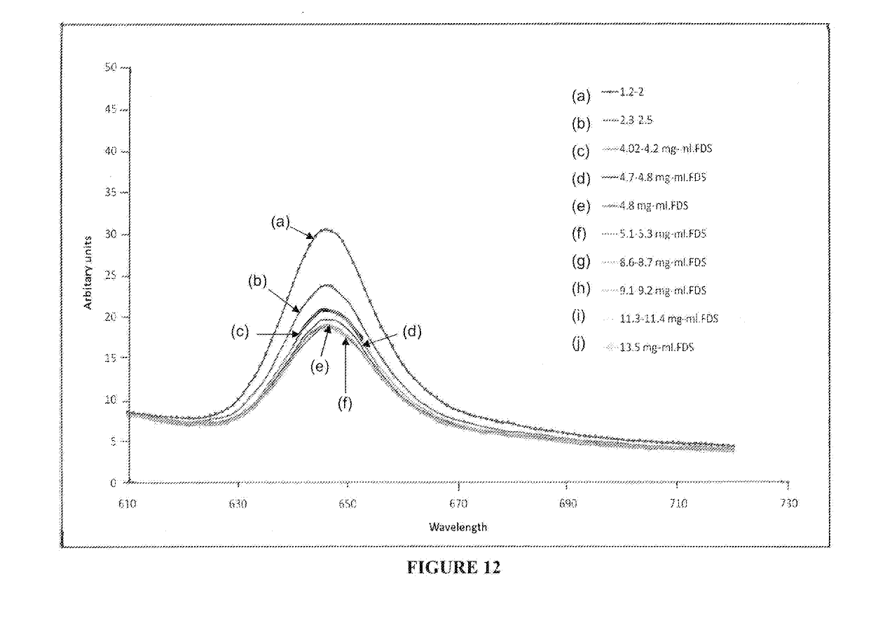

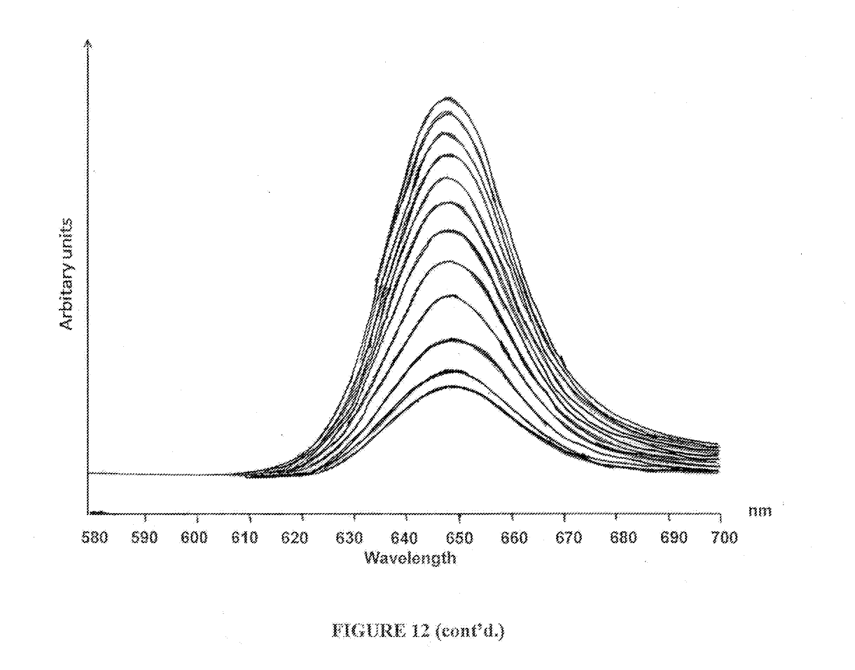

Oxygen sensing experiments using PtOEP loaded nanoparticles, and the nano-in micro-matrix showed no significant differences in the oxygen diffusion patterns (FIG. 10). Ksv value calculated from the slope of the stern volmer plot of I₀/I Vs concentration indicated no significant differences. Ksv value shows the oxygen diffusion behaviour in the matrix. Both the nanoparticles and nano-in-micro system showed linearity in the range of 0-6 mg/L with a good regression coefficient. The dynamic response with reversibility was also determined using nanoparticles and nano-in-micro system (FIG. 11). Both the systems were found to be reversible in nature with changing concentration of O₂. An overlay of fluorescene emission of PtOEP in response to varying concentrations of O₂ is shown in (FIG. 12 a).

### Example 8

### In-Vitro Lactate Sensing

500 μl of nano-in-micro particles containing dye and lactate oxidase was mixed with fixed concentration of Sodium Lactate in the range of 0-20 mM. The suspension was mixed and fluorescence emission scans were obtained at an excitation of 530 nm and emission of 645 nm. The maximum intensity was plotted against concentration and a calibration curve prepared.

FIG. 12 (b) indicates that fluorescence emission intensity for different concentrations of sodium lactate in the range of 0-20 mM, with higher concentrations showing an increase in emission intensity.

### Example 9

### In-Vitro Lactate Sensing

Several parameters like reproducibility, accuracy, precision, reversibility, interday and intraday variability, linearity, range and the regression coefficient were calculated.

### Example 10

### In Vivo Detection and Measurement

Nano-in-micro matrices are made in accordance with the above Examples.

In one example, the nano-in-micro matrices are injected into a subject (for example 1 ml, 5 ml, 10 ml of a solution at 1.5 mg/ml) intramuscularly at a depth of 3 mm. Chemiluminescent images are then captured using an IVIS imaging system (Xenogen, USA) with a one minute acquisition time. The amount of chemiluminescence is used to calculate the amount of lactate, pyruvate, oxygen or hydrogen peroxide in the muscle of the subject; which in turn, is used as a marker for metabolic status in the subject.

### REFERENCES

1. Lee D, Khaja S, Velasquez-Castano J C, Dasaril M, Sun C, Petros J, Taylor W R and Murthy N (2007) In vivo imaging of hydrogen peroxide with chemiluminescent nanoparticles. Nature letters 1-5
2. Araki N, Amao Y, Funabiki T, Kamitakahara M, Ohtsuki C, Mitsuo K, Asai K, Obata M and Yano S (2007) Optical oxygen-sensing properties of porphyrin derivatives anchored on ordered porous aluminium oxide plates. Photochemical & photobiological sciences: Official journal of the European Photochemistry Association and the European Society for Photobiology 6: 794-803
3. Basu B J (2007) Optical oxygen sensing based on luminescence quenching of platinum porphyrin dyes doped in ormosil coatings. Sensors & Actuators: B. Chemical 123: 568-577
4. Oter O, Ertekin K, Dayan O and Cetinkaya B (2008) Photocharacterization of novel ruthenium dyes and their utilities as oxygen sensing materials in presence of perfluorochemicals. Journal of fluorescence 18: 269-276
5. Oter O, Ertekin K and Derinkuyu S (2009) Photophysical and optical oxygen sensing properties of tris(bipyridine)ruthenium(II) in ionic liquid modified sol-gel. Materials Chemistry & Physics 113: 322-328
6. De Vos P, De Haan B J and Van Schilfgaarde R (1997) Upscaling the production of microencapsulated pancreatic islets. Biomaterials 18: 1085-1090
7. Kwok K K, Groves M J and Burgess D J (1991) Productin of 5-15 micron diameter alginate-polylysine microcapsules by an air atomization technique Pharmaceutical Research 8: 340-44
8. Ribeiro C C, Barrias C C and Barbosa M A (2004) Calcium phosphate-alginate microspheres as enzyme delivery matrices. Biomaterials 25: 4363-4373
9. Su J, Tao X, Xu H and Chen J F (2007) Facile encapsulation of nanoparticles in nanoorganized bio-polyelectrolyte microshells. Polymer 48: 7598-7603
10. www.nanotechbriefs.com, Accessed on 26 Oct. 2008.
11. http://www.johnmorris.com.au/html/Ysi/ysi1500.html, Accessed on 26 Oct. 2008.
12. http:www.fitnessmonitors.com/ecstore/cat111.html, Accessed on 26 Oct. 2008.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 particles refers to groups having 1, 2, or 3 particles. Similarly, a group having 1-5 particles refers to groups having 1, 2, 3, 4, or 5 particles, and so forth. As used herein, the term “about” means in quantitative terms, plus or minus 10%.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of measuring lactate, pyruvate, oxygen or hydrogen peroxide levels in a subject; comprising:
implanting in the subject a biosensor comprising a nano-in-micro matrix, wherein the nano-in-micro matrix is used to measure the lactate, pyruvate, oxygen or hydrogen peroxide levels.

2. The method of claim 1, wherein the size of the nanoparticles of the nano-in-micro matrix are between 1 and 2000 nm and wherein the microparticles are between 650 nm and 100 μm.

3. The method of claim 1, wherein the nanoparticles of the nano-in-micro matrix comprise a copolymer synthesized from one or more of: 4-hydroxyl benzyl alcohol, 1,8-octane diol, polyethylene imine, or oxalyl chloride.

4. The method of claim 1, wherein the implanting comprises injecting the biosensor intramuscularly, intravenously, subcutaneously, intradermally or intraperitoneally in the subject.

5. The method of claim 1, wherein the biosensor comprises lactate oxidase (Lox), lactate dehydrogenase, and/or lactate monooxygenase.

6. The method of claim 1, wherein the nano-in-micro matrix comprises a peroxalate polymer that emits light in the near infra red range (600 nm to 1100 nm) in the presence of hydrogen peroxide.

7. The method of claim 6, wherein the method further comprises detecting light at 630 nm emitted from a fluorophore in the nano-in-micro matrix, and correlating the light to the presence and/or amount of hydrogen peroxide in the subject.

8. The method of claim 1, wherein the nano-in-micro matrix comprises at least one fluorescence based oxygen sensitive dye selected from: ruthenium based dyes or porphyrins.

9. The method of claim 1, wherein the method comprises measuring and/or detection of fluorescence and/or light emitted from the nano-in-micro matrix, and correlating the fluorescence and/or light to the presence and/or amount of hydrogen peroxide or oxygen in the subject.

10. The method of claim 9, wherein the amount of hydrogen peroxide or oxygen is used to calculate the amount of lactate in the subject.

11. The method of claim 1, wherein the nano-in-micro matrix is implanted intramuscularly into a subject at a depth of about 3 mm.

12. A composition comprising:
a nano-in-micro matrix, wherein the nano-in-micro matrix comprises a copolymer synthesized from one or more of: 4-hydroxyl benzyl alcohol, 1,8-octane diol, polyethylene imine, or oxalyl chloride.

13. The composition of claim 12, wherein the nano-in-micro matrix comprises at least one fluorescence based oxygen sensitive dye selected from ruthenium based dyes or porphyrins.

14. The composition of claim 12, wherein the nano-in-micro matrix comprises lactate oxidase (Lox), lactate dehydrogenase, and/or lactate monooxygenase.

15. The composition of claim 12, wherein the size of the nanoparticles of the nano-in-micro matrix are between 1 and 2000 nm and wherein the microparticles are between 650 nm and 100 μm.

16. The composition of claim 12, wherein the nano-in-micro matrix emits light in the near infra red range (600 nm to 1100 nm) in the presence of hydrogen peroxide.

17. The composition of claim 12, wherein the nano-in-micro matrix emits light in the near infra red range (600 nm to 1100 nm) in the presence of oxygen.

18. The composition of claim 12, wherein the nanoparticles and/or microparticles of the nano-in-micro matrix comprise one or more of PSS, PAH, PAA, chondroitin sulphate, collagen, dextran sulphate, PEI, chitosan, or alginate.

19. The composition of claim 1, wherein the nanoparticles and/or microparticles of the nano-in-micro matrix are coated using layer-by-layer self assembly with at least one polyelectrolyte.

20. A method of monitoring the metabolic status of a subject, comprising:
implanting in the subject a composition of claim 12; wherein the composition is used to measure the lactate, pyruvate, oxygen or hydrogen peroxide levels; and wherein the levels of lactate, pyruvate, oxygen or hydrogen peroxide levels are indicative of the metabolic status of the subject.
